(19) United States
(12) Patent Application Publication
(10) Pub. No.: US 2026/0354554 A1
(43) Pub. Date: Nov. 12, 2026

(54) HAIR AND SCALP APPLICATOR WITH FORMULA CLEANSING

(71) Applicant: L'Oréal, Paris (FR)

(72) Inventor: Dominic AKERELE, Brooklyn, NY (US)

(73) Assignee: L'Oréal, Paris (FR)

(21) Appl. No.: 19/725,699

(22) Filed: Jun. 30, 2026

(51) Int. Cl. A45D 19/02; A61K 8/34; A61K 8/41; A61K 8/44; A61K 8/49; A61K 8/73; A61K 8/891; A61K 8/892; A61K 8/894; A61K 8/896; A61Q 5/02

(52) U.S. Cl. CPC A45D 19/028; A61K 8/34; A61K 8/345; A61K 8/411; A61K 8/44; A61K 8/442; A61K 8/4973; A61K 8/735; A61K 8/891; A61K 8/892; A61K 8/894; A61K 8/896; A61Q 5/02; A45D 2200/056; A45D 2200/207

(57) ABSTRACT

A system for treating a scalp, hair, or a combination thereof, the device including a formula comprising at least 25% alcohol, and an applicator, including a dispense pump configured for depositing the formula, a dispense port configured to dispense the formula, a formula channel configured for fluidly coupling the dispense pump and the dispense port, a transducer configured for emitting ultrasonic energy, at least one vacuum inlet disposed around the applicator, configured for applying suctioning, a vacuum pump configured to apply suction, and a vacuum channel configured for fluidly coupling the plurality of vacuum inlets to a waste reservoir.

### SUMMARY

In one aspect, disclosed herein is a system for treating a scalp, hair, or a combination thereof, the device including a formula comprising at least 25% alcohol, and an applicator, including a dispense pump configured for depositing the formula, a dispense port configured to dispense the formula, a formula channel configured for fluidly coupling the dispense pump and the dispense port, a transducer configured for emitting ultrasonic energy, at least one vacuum inlet disposed around the applicator, configured for applying suctioning, a vacuum pump configured to apply suction, and a vacuum channel configured for fluidly coupling the plurality of vacuum inlets to a waste reservoir.

In some embodiments, the formula comprises up to 40% alcohol.

In some embodiments, the formula further includes water and an anionic surfactant. In some embodiments, the formula includes about 70% water. In some embodiments, the anionic surfactants are selected from disodium cocoyl glutamate, sodium cocoyl glutamate, sodium lauryl sulfate, sodium laureth sulfate, ammonium lauryl sulfate, linear alkylbenzene sulfonate, alpha olefin sulfonate, sodium dodecyl benzene sulfonate, or combinations thereof.

In some embodiments, the formula further comprises one or more active ingredients. In some embodiments, the one or more active ingredients are selected from brassicamidopropyl dimethylamine, sodium hyaluronate, hyaluronic acid, glycerin, polyvinylpyrrolidone, niacinamide, piroctone olamine, ginger root extract, lactic acid, and combinations thereof. In some embodiments, the one or more active ingredients comprise silicones. In some embodiments, the silicones are selected from dimethicone copolyol, dimethicone PEG-8 phosphate, hydrolyzed silk PG-propyl methylsilanediol, lauryl methicone copolyol, PEG-8 dimethicone, and combinations thereof. In some embodiments, the silicones are selected from amodimethicone, bis-aminopropyl dimethicone, cetearyl methicone, cetyl dimethicone, cyclomethicone, cyclopentasiloxane, dimethicone, dimethiconol, phenyl trimethicone, stearyl dimethicone, trimethylsilylamodimethicone, and combinations thereof.

In some embodiments, the at least one vacuum inlet is a plurality of vacuum inlets.

In some embodiments, the formula is configured to reduce a cavitation threshold of sebum when applied to scalp, hair, or skin, and in response to the ultrasonic energy.

In some embodiments, the formula further includes a fragrance, wherein the fragrance is selected from paraphenylenediamine (PPD) fragrance, cyclopentadecanolide (CPD) fragrance, or a combination thereof.

In some embodiments, the formula is configured for cleansing skin, scalp, or hair, styling or volumizing hair, protecting color treated hair, controlling oil of hair, skin, or scalp, preventing or reducing dandruff, promoting hair growth, or a combination thereof.

In some embodiments, the formula includes about 70% water, about 25% alcohol, about 3% disodium cocoyl glutamate, sodium cocoyl glutamate, or a combination thereof, about 0.4% brassicaminopropyl dimethylamine, about 0.1% to about 0.2% sodium hyaluronate, and about 1% glycerin.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example device for treating a user's scalp and/or hair, in accordance with the present technology;

FIG. 1B is an example closeup of the device of FIG. 1A, in accordance with the present technology;

FIG. 1C is a left-hand view of the device of FIG. 1A, in accordance with the present technology;

FIG. 1D is a right-hand view of the device of FIG. 1A, in accordance with the present technology;

FIG. 2A is an example ultrasonic horn, in accordance with the present technology;

FIG. 2B is an example ceramic plate, in accordance with the present technology;

FIG. 3A is a block diagram of the internal components of an example device, in accordance with the present technology;

FIG. 3B is another example block diagram of an example device, in accordance with the present technology;

FIG. 4 is an example system, in accordance with the present technology;

FIGS. 5A-5B show example fluid channels of a device, in accordance with the present technology;

FIG. 6 is a graph showing the reduction of sebum achieved by the formula in combination with the disclosed device, in accordance with the present technology;

FIGS. 7A-7B are graphs showing the visual and tactile grease of hair before and after applying the disclosed formula in combination with the disclosed device, in accordance with the present technology;

FIG. 8 is an example method of using a device, in accordance with the present technology; and

FIG. 9 is yet another method of using a device, in accordance with the present technology.

### DETAILED DESCRIPTION

Disclosed herein are devices, systems, and methods for treating a scalp, hair, and/or skin. In some embodiments, the device described herein is configured to dispense a formula on the scalp/skin/hair (such as the roots of hair), emulsify the formula, and then suction waste material (including excess formula, dirt, dandruff, sebum, and the like). In some embodiments, the formula is a high-alcohol content formula (such as 25% alcohol or greater). In some embodiments, the formula, in combination with ultrasonic energy emitted by a transducer of the devices disclosed herein reduce a cavitation threshold of sebum, making it easier to remove the sebum and clean the scalp, during and after treatment.

The device may further include a cartridge for both containing the formula and receiving the waste material from the scalp. The device may include a number of control panels having actuators and light-emitting diodes (LEDs) for visually alerting a user as to when and whether the formula is being dispensed and/or suctioning is occurring. In some embodiments, additional energy therapies, such as light therapy, heat therapy, and ultrasonic therapy are delivered by the device. The resulting system is capable of efficiently and gently cleansing the scalp, hair and/or skin of a user.

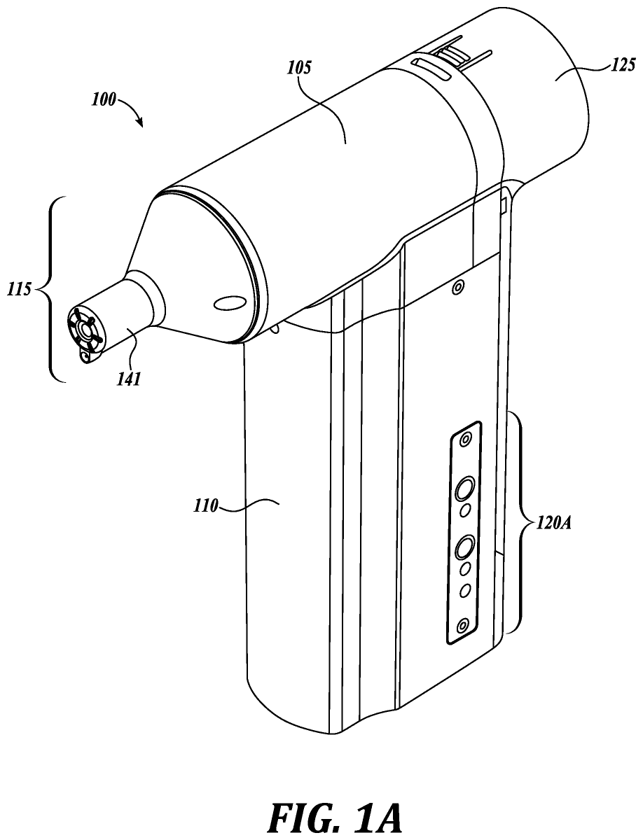

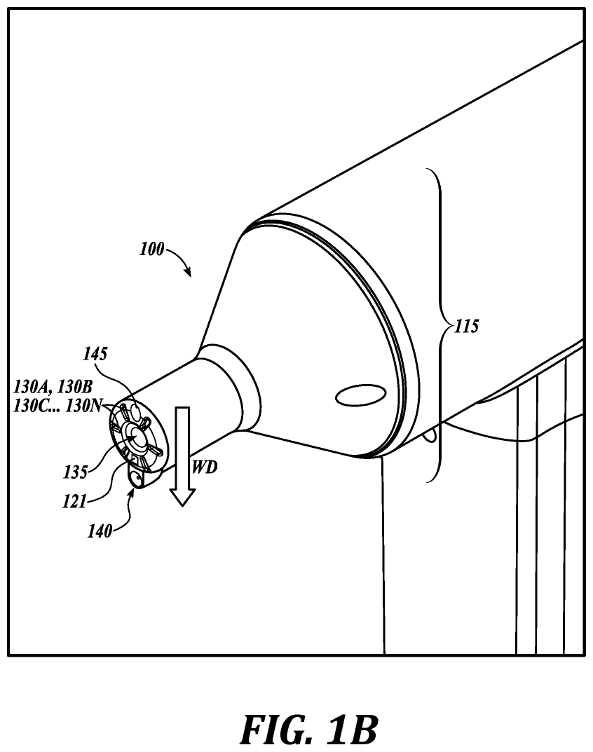

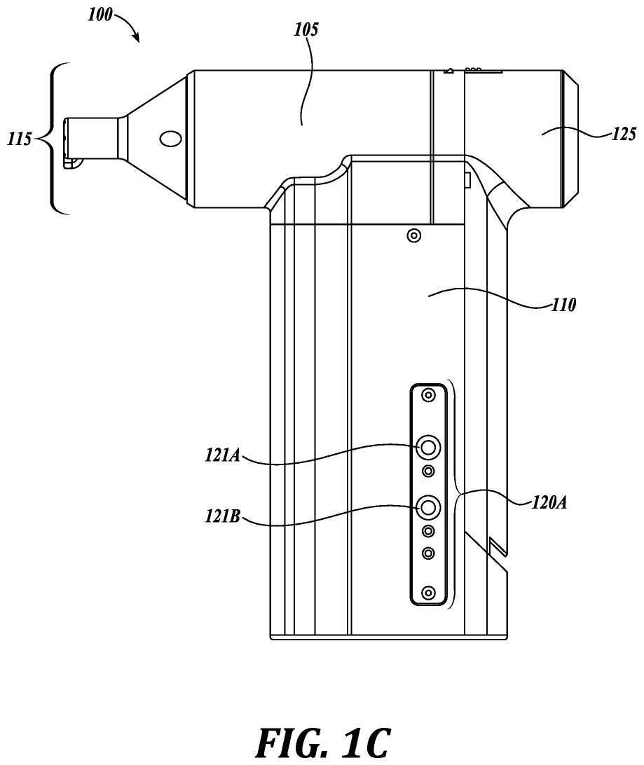

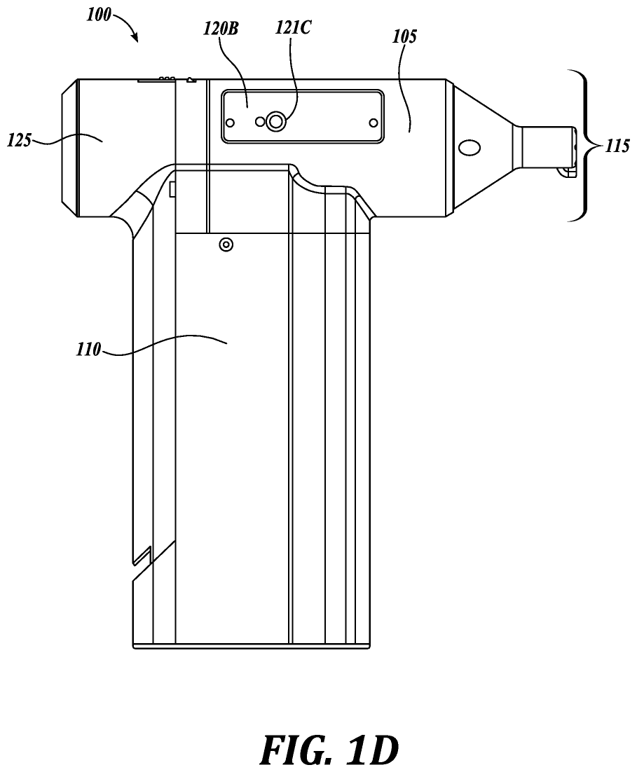

FIG. 1A is an example device 100 for treating a user's scalp and/or hair, in accordance with the present technology. In some embodiments, the device 100 includes a body 105 and a handle 110. In some embodiments, the body 105 includes an applicator 115 configured for dispensing a formula and/or applying one or more energy therapies, as shown and described herein. In some embodiments, the applicator 115 includes a heating element 141 configured to apply heat to a user's scalp, hair, or skin. In some embodiments, the heating element 141 may be omitted. In some embodiments, the device further includes a cartridge 125 configured for holding the formula and/or containing a waste material, as explained in detail herein. In some embodiments, the handle includes a first control panel 120. The first control panel 120 may include one or more buttons, actuators, capacitive touch type buttons, switches, or the like. In some embodiments, the first control panel 120 includes controls for the applicator 115 as explained herein.

In operation, the cartridge 125 may be removably coupled to the body 105 of the device. In some embodiments, the cartridge 125 includes a formula, such as a cleanser, a leave in shampoo, a leave-in conditioner, or the like. In some embodiments, the formula includes beta hydroxy acid (BHA). In some embodiments, the formula includes salicylic acid. In some embodiments, one or more actuators on the first control panel 120 are actuated to (1) turn on the device 100, (2) dispense the formula from the cartridge 125, (3) apply one or more energy therapies (such as ultrasonic therapy, heat therapy, light therapy, or the like), (4) suction waste product (including emulsified formula, sebum, dirt, and other contaminants) into the cartridge 125, or (5) a combination thereof.

FIG. 1B is an example closeup of the device 100 of FIG. 1A, in accordance with the present technology. In some embodiments, the body 105 of the device 100 had a diameter bd that is substantially equal to a diameter of the cartridge 125. In some embodiments, the device 100 includes an applicator 115 configured for dispensing the formula, suctioning waste product from the hair and/or scalp and applying an energy treatment. In some embodiments, the applicator 115 includes a temperature sensor 121, one or more vacuum inlets 130A, 130B, 130C . . . 130N, an ultrasonic horn 135, a dispense port 140, and/or a light source 145.

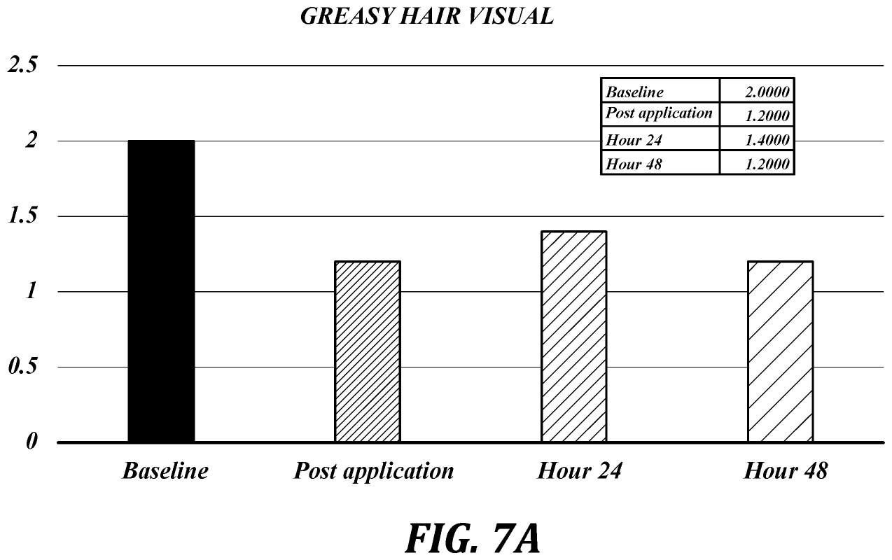

In some embodiments, the applicator 115 is configured for delivering a formula, such as from the cartridge 125 of FIG. 1A. In some embodiments, the formula is delivered from the dispense port 140 through one or more formula channels (as shown and described in detail in FIGS. 6A-6B. In some embodiments, the formula is a cleanser, a leave in shampoo, a leave in conditioner, a styling gel or cream, or the like.

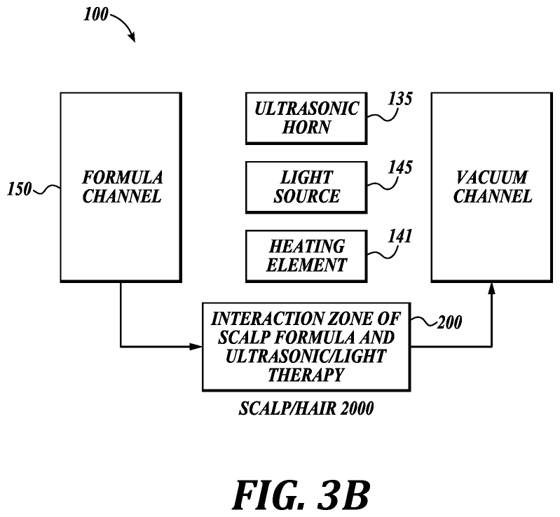

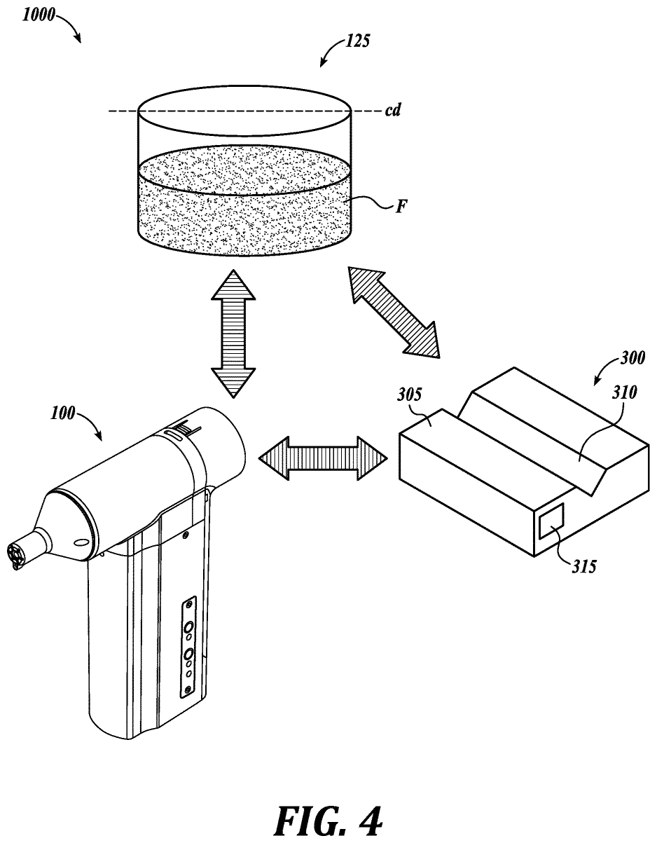

In some embodiments, the applicator 115 is configured to suction waste material from a user's hair, scalp, skin, or the like. In some embodiments, the waste material is sebum, emulsified formula, dirt, dandruff, contaminants, or the like. In some embodiments, the waste material is suctioned into the one or more vacuum inlets 130A, 130B, 130C . . . 130N through one or more vacuum channels (as shown and described in detail in FIGS. 6A-6B) and into the cartridge 125 of FIG. 1A. In some embodiments, the device 100 includes a vacuum pump (as shown and described in FIG. 3A) configured to suction the waste material into the cartridge 125.

In some embodiments, the applicator 115 is configured for delivering heat therapy (or “heat treatment”) with a heating element (such as heating element 141). In some embodiments, the heat treatment is configured to heat the scalp and/or hair to about 28 degrees Celsius to about 30 degrees Celsius. In some embodiments, the applicator further includes a temperature sensor 121. In some embodiments, the temperature sensor 121 is configured for measuring a temperature of the heating element 141 and/or a temperature of a user's scalp and/or hair. In some embodiments, the heating element 141 is disposed inside the applicator 115. In some embodiments, the heating element 141 is a nichrome wire. In some embodiments, the heating element 141 is a ceramic heating element. In some embodiments, the heating element 141 is a positive temperature coefficient (PTC) heater. In some embodiments, the heating element 141 is a radiant heating element. In some embodiments, the heating element 141 is an induction heating element.

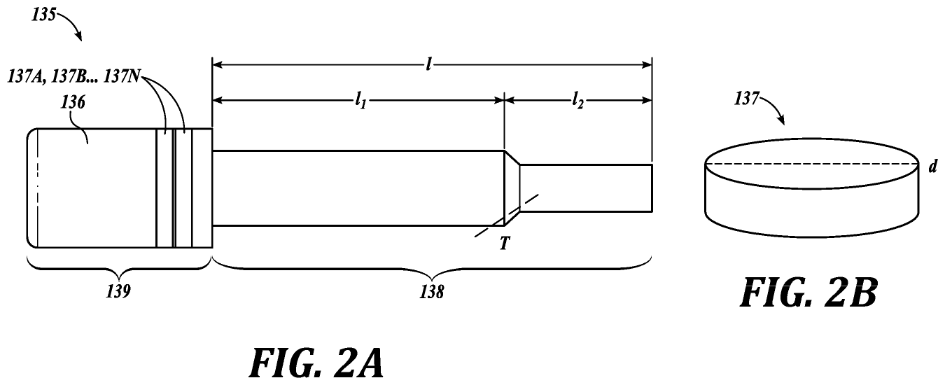

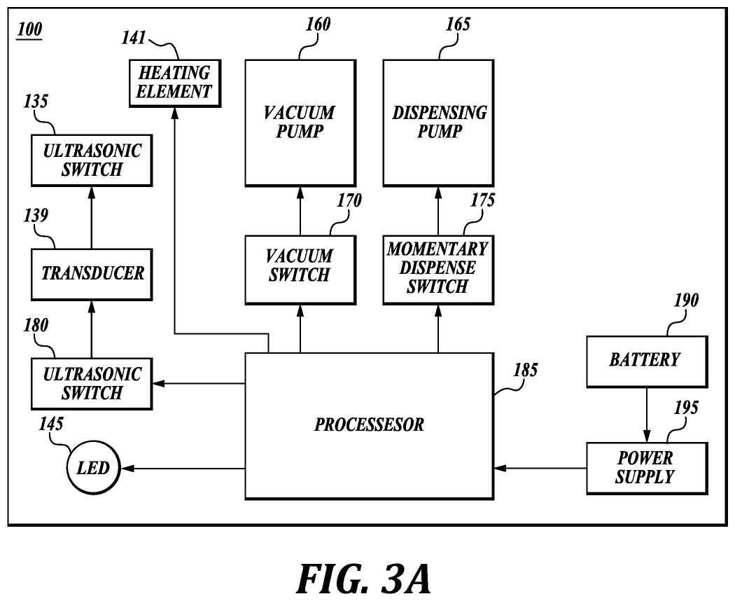

In some embodiments, the applicator 115 is configured for delivering ultrasonic therapy (or “ultrasonic treatment”) with the ultrasonic horn 135. In some embodiments, the ultrasonic treatment is configured for increasing one or more active ingredients in the formula. The ultrasonic horn is shown and described in detail in FIGS. 2A-2B. In some embodiments, the ultrasonic therapy is ultrasonic vibration. In some embodiments, a frequency of the ultrasonic vibration is between about 20 kHz to about 50 kHz. In some embodiments, the ultrasonic horn 135 is configured for precisely delivering ultrasonic energy to a concentrated location on the body (such as the skin, hair, and/or scalp) to achieve cavitation of a dispensed cleansing formula, effectively emulsifying the formula with sebum (naturally produced from sebaceous glands across the human body) and impurities (such as dirt, bacteria, dandruff, and the like). Simultaneously or contemporaneously, a vacuum shroud (Such as plurality of vacuum inlets 130A, 130B, 130C . . . 130C) collects the emulsified mixture, which is easily suctioned after ultrasonic cavitation, ensuring thorough removal. This results in the hair, scalp, and/or skin being cleaned. Ultrasonic energy not only allows for efficient emulsification of the formula, but also generates air bubbles (foaming effect) and atomization of the emulsified mixture, which enables efficient removal via the vacuum pump (such as vacuum pump 165).

In some embodiments, the ultrasonic horn 135 enables highly efficient cleansing of the human body (skin, scalp, and hair) via ultrasonic energy-induced cavitation, a key component that allows for highly efficient cleansing of the human body via cavitation, dispersion, and emulsification of the dispensed formula with sebum. These effects may allow a high level of cleanliness to be achieved that surpasses traditional methods. Deep removal of naturally produced oils (sebum) and collected dirt and debris, along with deep penetration of scalp and skin actives from the cleansing formula, driven deeper into pores and hair follicles than manual scrubbing, allows for a cleaner scalp without irritation or aggressive scrubbing. Further, the ultrasonic horn 135 may be used for targeted cleansing of hair, scalp, and skin to reduce the need for washing with water and extend the time between wash days. This targeted cleansing is less abrasive than manual scrubbing or physical exfoliation, enabling minimized irritation for sensitive skin and scalp. In some embodiments, the ultrasonic therapy results in a foaming effect (or generated air bubbles) within the emulsified mixture via ultrasonic cavitation, enabling effective removal via vacuum (suction). In some embodiments, the ultrasonic therapy may result in atomization of emulsified mixture, allowing for displacement of formula, sebum, and impurities from a substrate (i.e., hair, skin, or scalp), enabling more effective removal via the vacuum pump 165.

In some embodiments, the ultrasonic horn 135 is specifically designed and tuned to achieve cavitation of formula, sebum, and debris, across a range of targeted frequencies and allows for gentle and effective cleansing of the body. In some embodiments, the ultrasonic horn 135 is slightly inset from the applicator 115 such that the horn tip (as shown in FIG. 2A) is not in direct contact with the scalp, minimizing damping of the ultrasonic waves and ensuring safety of ultrasonic use. In some embodiments, the ultrasonic horn 135 contacts only the dispensed cleansing formula to induce cavitation that allows for emulsification of the sebum and debris on the body, aeration of the emulsified mixture, generating foam (air bubbles in the emulsified mixture) and atomization (emulsified mixture turns to mist that is propelled away from the substrate) that is more easily removed via the vacuum. In some embodiments, the ultrasonic transducer (as shown in FIG. 2B) produces ultrasonic waves at the resonant frequency of the ultrasonic horn 135 In operation, driving this frequency enables cavitation of the cleansing formula which results in effective emulsion. The shape of the ultrasonic horn 135 (as shown and described in FIGS. 2A-2B) is designed to place the resonant nodes at its tip (such as tip 138), i.e., the closest point to the skin/scalp/hair roots contact point, maximizing cavitation near the skin/scalp/hair roots surface.

The ultrasonic treatment disclosed herein results in a variety of advantages. For example, the ultrasonic treatment results in cleansed hair, roots, scalp, and/or skin, and dry and refreshed hair, roots, scalp, and/or skin, with removed/reduced sebum and debris. This ultrasonic treatment is also minimally irritating. The hair, skin, and scalp care ingredients in the deposited formula and remain after cleansing. Additional benefits include enhanced cleansing performance beyond standalone product formulas for the removal of sebum and debris (especially deeply lodged impurities), enhanced cleansing performance beyond traditional methods of hair, scalp, and body cleansing (via manual scrubbing), enhanced penetration of beneficial active ingredients from the cleansing formula into the hair follicles and skin pores, efficient removal of emulsified formula, sebum, and debris to leave minimal wetting and leftover sebum/debris via ultrasonic cavitation, reduced need for full washes with water, and extended time between traditional washes, and gentler cleansing of skin and scalp than manual/mechanical scrubbing, minimizing potential irritation, which is particularly beneficial for sensitive skin.

In some embodiments, the applicator 115 is configured to delivering light therapy (or “light treatment”) with the light source 145. The scalp microbiome plays a crucial role in maintaining the skin's barrier function, protecting against water loss and environmental stressors while inhibiting the overgrowth of harmful pathogens. An imbalance in this microbiome, often characterized by an overgrowth of Malassezia globosa (a fungus associated with dandruff), can lead to decreased barrier function, increased inflammation (e.g., excess sebum production), and dehydration, manifesting as dandruff and scalp irritation. Further exacerbating these issues is the common practice of over-washing the hair and scalp with harsh cleansers, stripping the scalp of its natural oils and disrupting the microbial balance. In some embodiments, the light source 145 is a light emitting diode (LED). In some embodiments, the light therapy is antimicrobial. Specifically, in some embodiments, by applying light therapy to a user's scalp and/or hair, Malassezia globosa may be reduced.

In some embodiments, the light therapy is anti-inflammatory. In some embodiments, the light therapy is configured for interacting with the formula applied by the applicator 115. For example, in some embodiments, one or more active ingredients in the formula are configured to interact with the light therapy. In some embodiments, the formula becomes “active” (i.e., effective) when it comes in contact with the light therapy. In some embodiments, the light therapy may react with the formula to form one or more reactive oxygen species. In this manner, the light therapy may increase the effectiveness of the formula.

In some embodiments, the formula is a cleansing formula dispensed by device onto hair and scalp, containing a photosensitizing agent that, when exposed to its corresponding wavelength of light, becomes activated and produces reactive oxygen species that can destroy harmful pathogens and reduce inflammation. In some embodiments, the formula further includes scalp care actives that deposit onto the hair fibers and scalp during the cleansing process, further alleviating scalp issues. In some embodiments, additional light therapy may include several light wavelengths included individually or in combination including ultraviolet (UV) light (and particularly UVB), which are shown to have anti-inflammatory and immunomodulatory effects on skin, and also shown to inhibit the growth of Malassezia globosa, blue light, which shown to have antimicrobial properties, efficacy in reducing Malassezia growth, and red light/near-infrared (NIR) light to improve scalp health by reducing inflammation. In some embodiments, specific wavelengths of light activate the photosensitizer in the formula for optimal targeted effects.

In some embodiments, the light therapy may be combined with additional energy therapies as described herein. For example, in one embodiment, the gentle yet effective cleansing action of the device is achieved through a non-contact ultrasonic horn (such as ultrasonic horn 135) head and a surrounding vacuum shroud that interact with the scalp and dispensed cleansing formula. In some embodiments, the ultrasonic horn 135 is a metal horn applicator head configured for precisely delivering ultrasonic energy to a concentrated location on the head to achieve cavitation of the dispensed cleansing formula and enable emulsification with sebum from the hair and scalp. Simultaneously, or contemporaneously, the vacuum shroud (such as plurality of vacuum inlets 130A, 130B, 130C . . . 130N) efficiently collects the emulsified mixture, ensuring thorough removal to leave the hair and scalp clean. The resulting suction, formula application, light therapy, and ultrasonic therapy may result in rebalanced scalp microbiome, immediate and lasting reduction in severity of dandruff and scalp irritation, and cleansed hair roots and scalp, left dry and refreshed, with removed/reduced sebum, dirt, residual hair product. In this manner, use of the device 100 may result in significant and lasting reduction of Malassezia globosa and other potentially pathogenic microbes on scalp, and an increase in beneficial commensal bacteria (rebalanced scalp microbiome), and improved treatment of dandruff and scalp irritation beyond traditional dandruff shampoos/hair care.

In some embodiments, the light therapy is red light having a wavelength of about 620 nm to about 750 nm. In some embodiments, the light therapy is ultraviolet (UV) light having a wavelength of about 100 nm to about 400 nm. In some embodiments, the light therapy is UVB light having a wavelength of about 280 to 315 nm. In some embodiments, the light therapy is infrared (IR) light having a wavelength of about 780 nm to about 1 mm. In some embodiments, the light therapy is blue light having a wavelength of about 380 to 500 nm. In some embodiments, the light source 145 is configured for switching between one or more wavelengths of light therapy. For example, the light source 145 may be configured for emitting both blue light and IR light.

The device 100 is configured to move in a working direction WD. This working direction may enable formula to be dispensed prior to ultrasonic energy treatment, or additional therapies.

FIG. 1C is a left-hand view of the device 100 of FIG. 1A, in accordance with the present technology. In some embodiments, the handle 110 is configured to comfortably fit into the hand of a user. In some embodiments, the handle 110 is removably coupled to the body 105 of the device 100. In some embodiments, the cartridge 125 has a diameter substantially equal to a diameter of the body 105 (as shown in FIG. 1B).

In some embodiments, the handle 110 includes the first control panel 120. In some embodiments, the first control panel 120 includes a first actuator 121A and a second actuator 121B. In some embodiments, the first control panel 120 may further include one or more LEDs. The one or more LEDs may indicate if the device is on, if the device is applying formula, applying treatment, or a combination thereof. In some embodiments, the one or more LEDs may indicate a battery level, formula level, waste material level, or a combination thereof. In some embodiments, the first actuator 121A is configured for directing the applicator 115 to apply the energy therapy (such as ultrasonic therapy, light therapy, and/or heat therapy). In some embodiments, the second actuator 121B is configured for directing the applicator 115 to apply the suction and/or deposit the formula.

FIG. 1D is a right-hand view of the device 100 of FIG. 1A, in accordance with the present technology. In some embodiments, the device 100 further includes a second control panel 120. In some embodiments, the second control panel 120 is located on the body 105. In some embodiments, the second control panel 120 includes a third actuator 121C. In some embodiments, the third actuator 121C is configured for directing the applicator 115 dispensing the formula. In some embodiments, the second control panel 120 further includes one or more LEDs. In some embodiments, the one or more LEDs indicate a level of the formula, an alert to replace the cartridge 125, or the like.

FIG. 2A is an example ultrasonic horn 135, in accordance with the present technology. In some embodiments, the ultrasonic horn 135 includes a transducer 139. The transducer may include a back mass and one or more piezoceramic plates 137A, 137B, 137C . . . 137N. In some embodiments, the ultrasonic horn 135 further includes a tip 138. The tip 138 may have a taper T. In some embodiments, the tip is made of two segments: a first segment 11 and a second segment 12.

In some embodiments, the transducer 139 is a fractioning transducer. In some embodiments, the transducer is a Langevin transducer. In some embodiments, the transducer is a bending beam transducer. In some embodiments, the back mass 136 is configured for reflecting sound waves and/or distributing preload force of the transducer 139.

In some embodiments, a length of the tip 138 is defined in Equation 1:

l=l₁+c/4f

Where l is a length of the tip 138, c is sound propagation velocity through the ultrasonic horn 135 material in m/s, and f is a frequency.

In some embodiments, the transducer 139 includes one or more piezoceramic plates 137A, 137B, 137C . . . 137N. In some embodiments, the one or more piezoceramic plates 137A, 137B, 137C . . . 137N is between one and four piezoceramic plates.

In some embodiments, the transducer 139 has a power input that ranges from about 2 W to about 10 W when the transducer 139 is in operation, such as when the transducer 139 is applying ultrasonic therapy. In some embodiments, the transducer 139 has an impedance ranging from about 50 ohms to about 150 ohms.

FIG. 2B is an example piezoceramic plate 137, in accordance with the present technology. In some embodiments, each piezoceramic plate 137 has a diameter d of about 10 mm to about 25 mm.

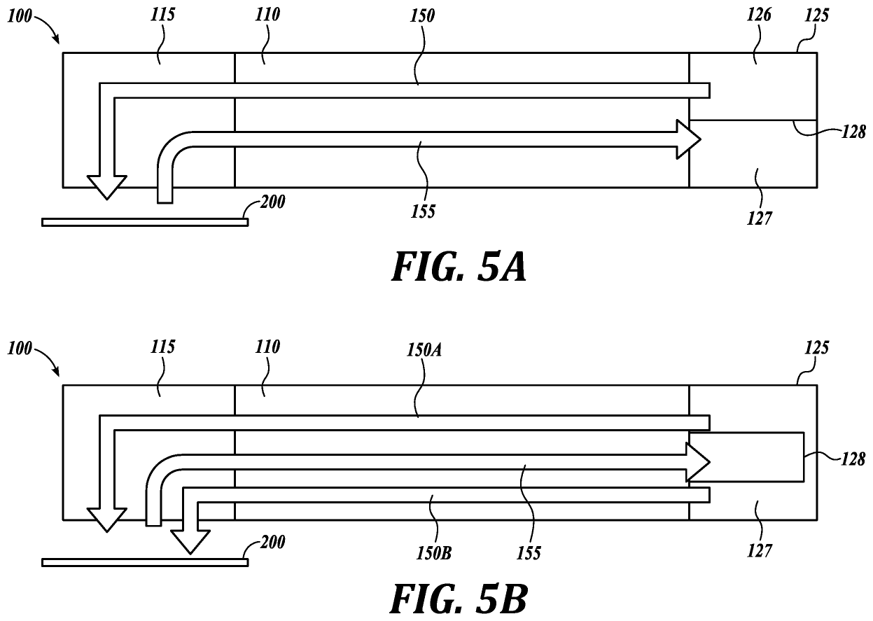

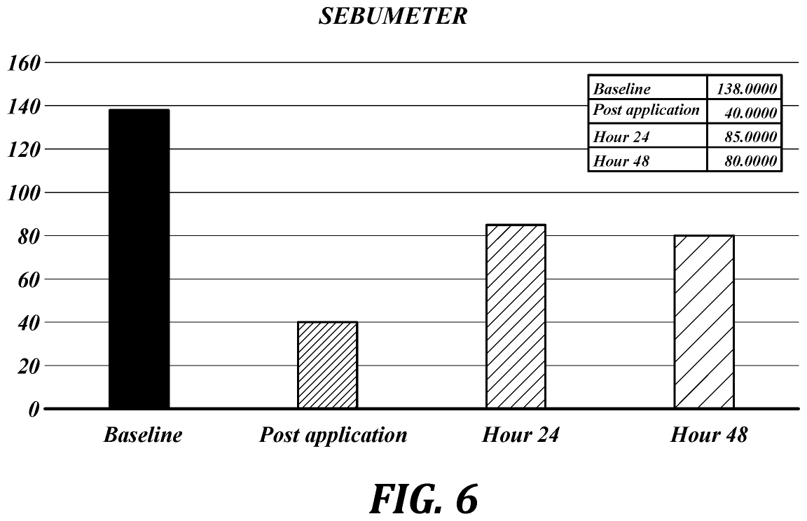

FIG. 3A is a block diagram of the internal components of an example device 100, in accordance with the present technology. In some embodiments, the device 100 includes a processor 185, a power supply 195, and a battery 190. In some embodiments, the processor 185 may direct any of the components described and shown in FIG. 3A. In some embodiments, the power supply 195 may power the processor 185 and one or more internal components of the device 100. In some embodiments, the battery 190 is a rechargeable battery, a capacitor, a single-use battery, or a capacitive touch type battery. In some embodiments, the device 100 may couple with a separate charger (as shown in FIGS. 4-5).

In some embodiments the device 100 further includes a vacuum pump 160, a vacuum switch 170, a dispense pump 165, and a dispense switch 175 (also referred to herein as a “momentary dispense switch”). In some embodiments, the vacuum pump 160 is configured to apply suction to a user's skin, scalp, or hair. The vacuum switch 170 may control the vacuum pump 160 when the vacuum switch 170 receives one or more commands from the processor 185. Similarly, the device 100 may include a dispense pump 165 configured to dispense a formula. The dispense switch 175 may control the dispense pump 165 when the dispense switch 175 receives one or more commands from the processor 185.

In operation, when a user presses an actuator on the device 100 (such as first actuator 121A, second actuator 121B, or third actuator 121C), the processor 185 may send one or more commands to the dispense switch 175 to direct the dispense pump 165 to dispense the formula. In some embodiments, when a user presses an actuator on the device 100 (such as first actuator 121A, second actuator 121B, or third actuator 121C), the processor 185 may send one or more commands to the vacuum switch 170 to direct the vacuum pump 160 to suction waste material from the user's hair, scalp, skin, etc.

In some embodiments, the device 100 is configured to applying an ultrasonic treatment. In some embodiments, the device 100 includes an ultrasonic horn 135, a transducer 139, and an ultrasonic switch 180. In some embodiments, when a user presses an actuator on the device 100 (such as first actuator 121A, second actuator 121B, or third actuator 121C), the processor 185 may send one or more commands to the ultrasonic switch 180 to direct the ultrasonic horn 135 to apply ultrasonic therapy with the transducer 139.

In some embodiments, the device 100 is configured to deliver a light therapy. In some embodiments, the device includes a light source 145. In some embodiments, the processor 185 may direct the light source 145 to turn the light source 145 on and off. In some embodiments, the processor 185 may adjust a brightness, color, or wavelength of the light source 145.

In some embodiments, the device 100 is configured to deliver a heat therapy. In some embodiments, the device 100 may further include a heating element 141 and/or a temperature sensor (as shown in FIG. 2B). In some embodiments, the processor 185 may direct the heating element 141 to turn on and off. In some embodiments, the processor 185 may further direct the heating element 141 to adjust a temperature of the heating element 141. In some embodiments, the processor 185 is further configured to set a temperature threshold for the heating element. By monitoring a temperature of the user's hair and/or scalp with a temperature sensor (such as temperature sensor 121), the processor may further direct the heating element to turn on or turn off. For example, if the temperature measured meets or exceeds the temperature threshold, the heating element 141 is turned off.

As shown in FIG. 3A, in some embodiments, the device 100 is configured to deliver heat therapy, ultrasonic therapy, and light therapy. It should be understood that in some embodiments, the device 100 may be configured for delivering only one or a subsection of these energy therapies. For example, in one embodiment, the device 100 is configured for delivering light therapy only. In another embodiment, the device 100 is configured for delivering light therapy and ultrasonic therapy.

FIG. 3B is another example block diagram of an example device 100, in accordance with the present technology. In some embodiments, the device 100 interacts with a user's scalp/hair 2000. Formula may be applied to the scalp/hair 2000 with one or more formula channels 150 of the device (as shown in detail in FIGS. 6A-6B). In some embodiments, the formula may then interact with one or more energy therapies of the device 100. For example, in some embodiments, the ultrasonic horn 135 may apply an ultrasonic therapy into an interaction zone of the scalp, formula, and energy therapy.

In some embodiments, after the formula interacts with the scalp and the energy treatment, any waste material (such as dirt, contaminants, sebum, and emulsified formula) may be suctioned into a cartridge of the device 100 through one or more vacuum channels 155 (as shown in detail in FIGS. 6A-6B).

FIG. 4 is an example system 1000, in accordance with the present technology. In some embodiments, the system 1000 includes the device 100 as shown and described herein, a cartridge 125, and a charger 300.

In some embodiments, the cartridge 125 contains a formula F. In dome embodiments, a diameter cd of the cartridge 125 is substantially equal to a diameter (such as diameter bd) of the body of the device 100. In some embodiments, the cartridge 125 is cylindrical. In some embodiments, the cartridge 125 is configured to couple with the device 100 such that the cartridge 125 is flush with the device 100 (or the body 105 of the device 100).

In some embodiments, the charger 300 includes a base 305, a charging interface 310, and a power source 315. In some embodiments, the charging interface 310 is configured for charging the device 100 through contact with the charging interface 310. In some embodiments, the charging interface 310 is a triangular groove. In some embodiments, the charging interface 310 is configured for removably coupling with the device 100. In some embodiments, the body (such as body 105) of the device 100 is configured for removably coupling the handle (such as handle 115). In such embodiments, the body 105 may be coupled to the charging interface 310. In some embodiments, the power source 315 is configured for enabling a wired connection to an outlet. In some embodiments, the power source 315 is a battery, such as a rechargeable battery.

FIGS. 5A-5B show example fluid channels of a device 100, in accordance with the present technology. In some embodiments, the device 100 includes one or more formula channels 150, 150A, 150B and one or more vacuum channels 155 (collectively “fluid channels”). In some embodiments, the cartridge 125 is fluidly coupled to the one or more formula channels 150, 150A, 150B of the device 100. Further, in some embodiments, the cartridge 125 is fluidly coupled to the one or more vacuum channels 155.

In some embodiments, the cartridge 125 includes a formula reservoir 126, a waste reservoir 127, and a partition 128 for separating the formula reservoir 126 and the waste reservoir 127. In some embodiments, the cartridge 125 is configured for removably coupling with the device 100 where when the cartridge 125 is coupled to the device, the formula reservoir 126 is fluidly coupled with the one or more formula channels 150, 150A, 150B of the device 100, and the waste reservoir 127 is fluidly coupled with a vacuum channel 155 of the device. In some embodiments, the waste reservoir 127 is larger in volume than the formula reservoir 126. For example, in some embodiments, the waste reservoir 127 is about 10% to about 15% larger in volume than the formula reservoir 126.

In some embodiments, as shown in FIG. 5A, the partition 128 is a middle partition. The middle partition may separate the formula reservoir 126 and the waste reservoir 127 may be of substantially equal size. In such embodiments, the device 100 may include a formula channel 150 and a vacuum channel 155. The formula may be dispensed onto the hair/scalp 200 through the formula channel 150. Waste materials, such as dirt, sebum, emulsified formula, and the like, may then be suctioned into the waste reservoir through the vacuum channel 155.

In some embodiments, as shown in FIG. 5B, the device 100 includes a first formula channel 150A and a second formula channel 150B. In some embodiments, the partition 128 of the cartridge 125 is a concentric partition. In some embodiments, the formula reservoir 126 is fluidly coupled to both the first formula channel 150A and the second formula channel 150B.

In one aspect, disclosed herein is a system for treating a scalp, hair, or a combination thereof, the device including a formula comprising at least 25% alcohol. In some embodiments, the formula further includes water, and an anionic surfactant. In some embodiments, the formula includes about 70% water. In some embodiments, the formula includes up to 40% alcohol.

In some embodiments, the anionic surfactants are selected from disodium cocoyl glutamate, sodium cocoyl glutamate, sodium lauryl sulfate, sodium laureth sulfate, ammonium lauryl sulfate, linear alkylbenzene sulfonate, alpha olefin sulfonate, sodium dodecyl benzene sulfonate, or combinations thereof.

The formula may also include one or more active ingredients.

In some embodiments, one or more active ingredients are selected from brassicamidopropyl dimethylamine, sodium hyaluronate, hyaluronic acid, glycerin, polyvinylpyrrolidone, niacinamide, piroctone olamine, ginger root extract, lactic acid, and combinations thereof. In some embodiments, the formula is configured to cavitate (i.e., produce air bubbles) in response to the ultrasonic energy.

In some embodiments, the formula further includes a fragrance, wherein the fragrance is selected from p-phenylenediamine (PPD) fragrance, cyclopentadecanolide (CPD) fragrance, or a combination thereof. In some embodiments, the fragrance is an eau de toilette, eau de parfum, parfum, or eau de cologne.

In some embodiments, the one or more active ingredients include silicones. In some embodiments, the silicones are selected from dimethicone copolyol, dimethicone PEG-8 phosphate, hydrolyzed silk PG-propyl methylsilanediol, lauryl methicone copolyol, PEG-8 dimethicone, and combinations thereof. In some embodiments, the silicones are selected from amodimethicone, bis-aminopropyl dimethicone, cetearyl methicone, cetyl dimethicone, cyclomethicone, cyclopentasiloxane, dimethicone, dimethiconol, phenyl trimethicone, stearyl dimethicone, trimethylsilylamodimethicone, and combinations thereof.

In some embodiments, the formula is configured for cleansing skin, scalp, or hair. In some embodiments, the formula is configured to styling or volumizing hair. In some embodiments, the formula is configured for protecting color treated hair. In some embodiments, the formula is configured for controlling oil of hair, skin, or scalp. In some embodiments, the formula is configured for preventing or reducing dandruff. In some embodiments, the formula is configured for promoting hair growth.

In one example, the formula includes about 70% water, about 25% water, about 3% disodium cocoyl glutamate, sodium cocoyl glutamate, or a combination thereof, about 0.4% brassicaminopropyl dimethylamine, about 0.1% to about 0.2% sodium hyaluronate, and about 1% glycerin. A representative formula is shown in Table 1.

TABLE 1

| Ingredient | % Concentration |
|---|---|
| water | 70.49 |
| alcohol | 25 |
| disodium cocoyl glutamate and sodium | 3 |
| cocoyl glutamate | |
| brassicaminopropyl dimethylamine | 0.4 |
| sodium hyaluronate | 0.11 |
| glycerin | 1 |

The formula in Table 1 may be referred to as “Formula 1” herein. Formula 1 exhibits many advantageous properties, including a reduction of sebum, a reduction of greasiness in hair (both visual and tactile), and a low displacement threshold for cavitation at a particular frequency (such as a frequency emitted by the device).

FIG. 6 is a graph showing the reduction of sebum achieved by the formula in combination with the disclosed device, in accordance with the present technology. As shown, using the device and formula significantly reduced the presence of sebum, even up to 48 hours after application.

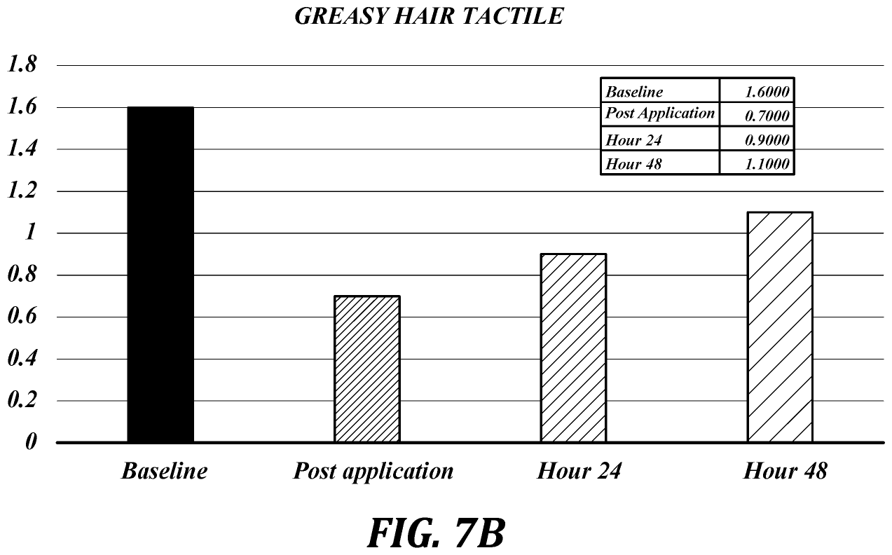

FIGS. 7A-7B are graphs showing the visual and tactile grease of hair before and after applying the disclosed formula in combination with the disclosed device, in accordance with the present technology. As shown, using the device and formula significantly reduced the feeling and look of greasiness of the hair, even up to 48 hours after application.

Table 2 shows the low displacement threshold for cavitation of the formula in comparison to other compositions, including tap water, typical water, clean water, degassed water, artificial sebum, and sebum mixed with Formula 1.

TABLE 2

| | | Speed of | Surface | Vapor | Cavitation |
|---|---|---|---|---|---|
| | Density | Sound | Tension | Pressure | Threshold |
| Material | (kg/m³) | (m/s) | (N/m) | (Pa) | (MPa) |
| Tap Water | 1000 | 1480 | 0.072 | 2330 | 1.5 |
| Typical Water | 1000 | 1480 | 0.072 | 2330 | 2 |
| Clean Water | 1000 | 1480 | 0.072 | 2330 | 3 |
| Degassed Water | 1000 | 1480 | 0.072 | 2330 | 5 |
| Formula 1 | 800 | 1300 | 0.035 | 5866 | 1.8 |
| Sebum (Artificial) | 930 | 1450 | 0.025 | 100 | 2.3 |
| Formula 1 + Sebum | 903 | 1270 | 0.034 | 5289.4 | 1.85 |
| (90/10) | | | | | |

As shown in Table 2, when Formula 1 was mixed with sebum, it reduced the cavitation threshold from 2.3 (sebum alone) to 1.85. Thus, the formula and sebum may be cavitated in response to the ultrasonic energy emitted by the transducer of the device. As explained herein, this cavitation emulsifies the sebum and formula, making it possible to remove the sebum and leftover formula with vacuum channels of the device.

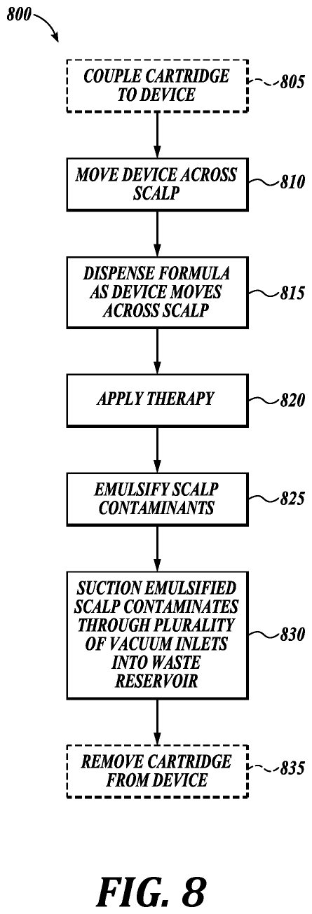

FIG. 8 is an example method 800 of using a device, in accordance with the present technology. In some embodiments, the method 800 is carried out with a device (such as device 100) including a dispense pump (such as dispense pump 165) configured to dispense a formula through a dispense port (such as dispense port 140) and a vacuum pump (such as vacuum pump 160). Optionally, the device may further include a light source (such as light source 145), an ultrasonic horn (such as ultrasonic horn 135), a heating element (such as heating element 141), a temperature sensor (such as temperature sensor 121), or a combination thereof.

In block 805, optionally, a cartridge (such as cartridge 125) is coupled to the device. In some embodiments, the cartridge includes a formula (such as formula F) and a waste reservoir (such as waste reservoir 127).

In block 810, the device is moved across a user's skin and/or scalp (such as skin/scalp 2000).

In block 815, the formula is dispensed as the device moves across the scalp. In some embodiments, the user may actuate an actuator (such as first actuator 121A, second actuator 121B, or third actuator 121C) of a control panel (such as first control panel 120A or second control panel 120B) to dispense the formula In some embodiments, the formula is dispensed from the cartridge through one or more formula channels (such as formula channels 150, 150A, 150B) and through the dispense port.

In block 820, therapy is applied. In some embodiments, therapy (or “energy therapy”) includes ultrasonic therapy, heat therapy, light therapy, or a combination thereof as described in detail herein. In some embodiments, the energy therapy is configured to react with the formula, such as emulsifying the formula, activating the formula, creating oxygen reactive species, or heating the formula, as described herein.

In block 825, scalp contaminants are emulsified. In some embodiments, scalp contaminants include dirt, sebum, dandruff, bacteria, and the like. The scalp contaminants may also be referred to as “waste material.”

In block 830, the waste material (or scalp contaminants) are suctioned into the cartridge through a plurality of vacuum inlets (such as vacuum inlets 130A, 130B, 130C . . . 130N). In some embodiments, the waste material is suctioned into the waste reservoir of the cartridge through one or more vacuum channels (such as vacuum channel 155) with the vacuum pump.

Optionally, in block 835, the cartridge may be removed from the device. In some embodiments, the cartridge may be cleaned and refilled with formula, to be used again. In some embodiments, the cartridge may be used multiple times before being removed from the device.

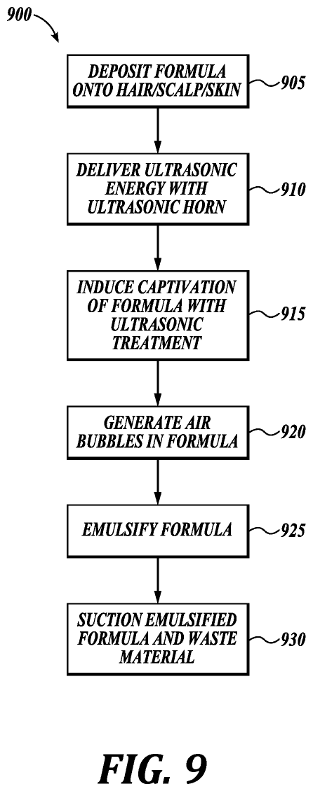

FIG. 9 is yet another method 900 of using a device, in accordance with the present technology. In some embodiments, the method 900 is carried out with a device (such as device 100) including a dispense pump (such as dispense pump 165) configured to dispense a formula through a dispense port (such as dispense port 140) and a vacuum pump (such as vacuum pump 160). Optionally, the device may further include a light source (such as light source 145), an ultrasonic horn (such as ultrasonic horn 135), a heating element (such as heating element 141), a temperature sensor (such as temperature sensor 121), or a combination thereof.

In block 905, formula is deposited onto the hair, the skin, the scalp, or a combination thereof. In some embodiments, the formula has one or more active ingredients activated by light therapy, ultrasonic therapy, or a combination thereof. In some embodiments, the formula is deposited through the dispense port from a cartridge (such as cartridge 125) through one or more formula channels (such as formula channels 150, 150A, 150B).

In block 910, ultrasonic energy is delivered with the ultrasonic horn. In some embodiments, the ultrasonic therapy is ultrasonic vibration. In some embodiments, a frequency of the ultrasonic vibration is between about 20 kHz to about 50 kHz. I

In block 915, cavitation of the formula is inducted with the ultrasonic treatment. In some embodiments, the ultrasonic horn 135 is configured for precisely delivering ultrasonic energy to a concentrated location on the body (such as the skin, hair, and/or scalp) to achieve cavitation of a dispensed cleansing formula, effectively emulsifying the formula with sebum (naturally produced from sebaceous glands across the human body) and impurities (such as dirt, bacteria, dandruff, and the like). As used herein, the term “cavitation” refers to the creation of microscopic bubbles in a liquid or formula. These bubbles rapidly expand and collapse, generating localized pressure and temperature fluctuations that can be leveraged for various applications, including chemical reactions and cell disruption. These bubbles form due to pressure variations created by the sound waves. When these bubbles collapse violently, they generate localized high temperatures and pressures, leading to a phenomenon called “micro-streaming” and generating reactive species with the formula.

In block 920, air bubbles are generated in the formula, also referred to herein as the “foaming effect.” In some embodiments, the ultrasonic treatment can significantly impact the foaming properties of formulas, often enhancing foaming capacity and stability by modifying protein structure and interactions at the air-water interface. In some embodiments, ultrasound can improve a protein's ability to create and stabilize foam, due to protein denaturation and unfolding (ultrasound can disrupt protein structures, exposing hydrophobic regions and increasing their ability to adsorb at the air-water interface, which is crucial for foam formation), reduced particle size (ultrasound can break down large protein aggregates, increasing the surface area available for interaction with air and improving foam formation), and improved solubility (ultrasound can enhance the solubility of proteins, making them more readily available to form a stable foam). This foaming effect can result in active ingredients in the formula penetrating farther into the skin barrier, delivering more active ingredients to the scalp and skin.

In block 925, the formula is emulsified. In some embodiments, ultrasonic treatment can be a highly effective for creating and stabilizing emulsions, particularly nano-emulsions, due to its ability to generate cavitation bubbles that break down liquids into tiny droplets. This process often leads to smaller droplet sizes, increased stability, and enhanced emulsification properties. Advantageously, emulsification of the formula is highly efficient, requiring relatively low energy input compared to other methods like high-pressure homogenization. Further, ultrasonic energy can produce emulsions with very small droplet sizes, even in the nanoscale, which can be crucial for certain applications. Ultrasonic treatment can improve the stability of emulsions, making them less prone to creaming, sedimentation, or coalescence.

Optionally, additional energy treatment, such as light therapy and/or heat therapy may also be delivered to the formula simultaneously with, or sequentially with the ultrasonic treatment. In some embodiments, the light therapy and/or heat therapy may further increase the emulsification of the formula. In some embodiments, the light therapy may further reduce inflammation, bacteria, fungus, dandruff, and other conditions of the skin, hair, and/or scalp.

In block 930, the emulsified formula and additional waste material (such as dirt, bacteria, fungus, dandruff, and other scalp contaminants) are suctioned into the cartridge. In some embodiments, the formula and waste material is suctioned through a plurality of suction inlets (such as plurality of suction inlets 130A, 130B, 130C . . . 130N) through one or more vacuum channels (such as vacuum channel 155).

It should be understood that all methods 800 and 900 should be interpreted as merely representative. In some embodiments, process blocks of all methods 800 and 900 may be performed simultaneously, sequentially, in a different order, or even omitted, without departing from the scope of this disclosure.

While illustrative embodiments have been illustrated and described, it will be appreciated that various changes can be made therein without departing from the spirit and scope of the invention.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed

What is claimed is:

1. A system for treating a scalp, hair, or a combination thereof, the device comprising:
a formula comprising at least 25% alcohol; and
an applicator, comprising:
a dispense pump configured for depositing the formula;
a dispense port configured to dispense the formula;
a formula channel configured for fluidly coupling the dispense pump and the dispense port;
a transducer configured for emitting ultrasonic energy;
at least one vacuum inlet disposed around the applicator, configured for applying suctioning;
a vacuum pump configured to apply suction; and
a vacuum channel configured for fluidly coupling the plurality of vacuum inlets to a waste reservoir.

2. The system of claim 1, wherein the formula comprises up to 40% alcohol.

3. The system of claim 1, wherein the formula further comprises:
water; and
an anionic surfactant.

4. The system of claim 3, wherein the formula comprises about 70% water.

5. The system of claim 3, wherein the anionic surfactants are selected from disodium cocoyl glutamate, sodium cocoyl glutamate, sodium lauryl sulfate, sodium laureth sulfate, ammonium lauryl sulfate, linear alkylbenzene sulfonate, alpha olefin sulfonate, sodium dodecyl benzene sulfonate, or combinations thereof.

6. The system of claim 1, wherein the formula further comprises one or more active ingredients.

7. The system of claim 6, wherein the one or more active ingredients are selected from brassicamidopropyl dimethylamine, sodium hyaluronate, hyaluronic acid, glycerin, polyvinylpyrrolidone, niacinamide, piroctone olamine, ginger root extract, lactic acid, and combinations thereof.

8. The system of claim 6, wherein the one or more active ingredients comprise silicones.

9. The system of claim 8, wherein the silicones are selected from dimethicone copolyol, dimethicone PEG-8 phosphate, hydrolyzed silk PG-propyl methylsilanediol, lauryl methicone copolyol, PEG-8 dimethicone, and combinations thereof.

10. The system of claim 8, wherein the silicones are selected from amodimethicone, bis-aminopropyl dimethicone, cetearyl methicone, cetyl dimethicone, cyclomethicone, cyclopentasiloxane, dimethicone, dimethiconol, phenyl trimethicone, stearyl dimethicone, trimethylsilylamodimethicone, and combinations thereof.

11. The system of claim 1, wherein the at least one vacuum inlet is a plurality of vacuum inlets.

12. The system of claim 1, wherein the formula is configured to reduce a cavitation threshold of sebum when applied to scalp, hair, or skin, and in response to the ultrasonic energy.

13. The system of claim 1, wherein the formula further includes a fragrance, wherein the fragrance is selected from paraphenylenediamine (PPD) fragrance, cyclopentadecanolide (CPD) fragrance, or a combination thereof.

14. The system of claim 1, wherein the formula is configured for cleansing skin, scalp, or hair, styling or volumizing hair, protecting color treated hair, controlling oil of hair, skin, or scalp, preventing or reducing dandruff, promoting hair growth, or a combination thereof.

15. The system of claim 1, wherein the formula comprises:
about 70% water;
about 25% alcohol;
about 3% disodium cocoyl glutamate, sodium cocoyl glutamate, or a combination thereof;
about 0.4% brassicaminopropyl dimethylamine;
about 0.1% to about 0.2% sodium hyaluronate; and
about 1% glycerin.
